(19) United States
(12) Patent Application Publication
(10) Pub. No.: US 2009/0221808 A1
(43) Pub. Date: Sep. 3, 2009

(54) Process for the Preparation of Nucleic Acid Duplexes

(76) Inventor: Michael J. Fazio, Midland, MI (US)

Correspondence Address: AGILENT TECHNOLOGIES INC., INTELLECTUAL PROPERTY ADMINISTRATION,LEGAL DEPT., MS BLDG. E P.O. BOX 7599, LOVELAND, CO, 80537, US

(21) Appl. No.: 12/092,942

(22) PCT Filed: Nov. 3, 2006
(86) PCT No.: PCT/US2006/043155

§ 371 Date: Sep. 29, 2008

Related U.S. Application Data
(60) Provisional application No. 60/734,364, filed on Nov. 7, 2005.

(51) Int. Cl. C07H 1/00

(52) U.S. Cl. 536/25.3

(57) ABSTRACT

The invention is a process for the preparation of at least one nucleic acid duplex by the steps of: (a) synthesizing a first single strand oligonucleotide to produce a crude solution of the first single strand oligonucleotide; (b) purifying the first single strand oligonucleotide from the crude solution by first liquid chromatography to produce a solution of purified first single strand oligonucleotide in a first liquid chromatography eluant; (c) synthesizing a second single strand oligonucleotide to produce a crude solution of the second single strand oligonucleotide; (d) purifying the second single strand oligonucleotide from the crude solution by second liquid chromatography to produce a solution of purified second single strand oligonucleotide in a second liquid chromatography eluant; (e) mixing the solution of purified first single strand oligonucleotide in the first liquid chromatography eluant with the solution of purified second single strand oligonucleotide in the second liquid chromatography eluant so that the first and second single strand oligonucleotides can complex to form a nucleic acid duplex in the mixture of first and second liquid chromatography eluants.

### BACKGROUND OF THE INVENTION

The present invention is in the field of nucleic acid duplexes and processes for the preparation of nucleic acid duplexes.

### BACKGROUND OF THE INVENTION

As discussed in US Patent Publication 2005/0037370, nucleic acid duplexes are well known in the pharmaceutical field. Example 236 of US Patent Publication 2005/0037370 describes a specific nucleic acid duplex and its preparation. As a further teaching in the art, Tuschl et al., Genes & Development 13; 3191-3197 (1999) describes the use and preparation of other nucleic acid duplexes.

Nucleic acid duplexes are conventionally prepared by mixing purified “sense” and “antisense” single strand oligonucleotides so that they can complex via formation of hydrogenation bonds between base pairs. The oligonucleotide may be natural or synthetically modified, ribo or deoxyribonucleic acid structure. Modifications can be to the bases (for example diaminopurine or 7-deaza-guanosine or the sugar moiety ex 2′-O-methyl, 2′-fluoro, or 2′-O-methoxyethyl) and/or to the internucleotide linkages (for example phosphodiester, phosphorothioate, phosphoramidate or phosphorothioamidate linkage or mixtures of these linkages). The single strand oligonucleotides can optionally be capped or conjugated. Modifications of oligonucleotides are well known in the art, see, for example and without limitation thereto, Manoharan, Current Opinion in Chemical Biology 2004, 8:570-579.

Single strand oligonucleotides for the production of nucleic acid duplexes are conventionally produced by the following procedure: (a) solid phase synthesis to produce a crude mixture of a single strand oligonucleotide; (b) liquid chromatography of the crude mixture to produce a solution of purified single strand oligonucleotide in the liquid chromatography eluant; (c) dialysis and/or ultrafiltration of the solution of purified single strand oligonucleotide in the liquid chromatography eluant to produce a desalted aqueous solution of the purified single strand oligonucleotide; and (d) freeze drying of the aqueous solution of purified single strand oligonucleotide to produce a solid consisting essentially of purified single strand oligonucleotide. Freeze drying of the aqueous solution of purified single strand oligonucleotide minimizes risk of microbial contamination and the generation of endotoxins during storage of the oligonucleotide. The process of duplex formation from two complementary single strand oligonuclcotides conventionally involves the steps of dissolving each isolated purified single strand oligonucleotide in water or buffered water solution. The two solutions of single strand oligonucleotide are then combined in the appropriate proportions to form the duplex. The solution can then be heated and cooled to insure the proper annealing of the duplex. If no salts are used in the process, the solutions are then freeze dried to isolate the desired duplex. If salts are used in the process the solution is desalted by ultrafiltration prior to freeze drying and isolation.

Although the conventional methods for preparing purified nucleic acid duplexes work adequately for small scale research purposes, such conventional methods also suffer from a number of problems when applied to larger scale production. For example, dialysis and/or ultrafiltration of the solution of each single strand oligonucleotide in the liquid chromatography eluant to produce an aqueous solution of the single strand oligonucleotide followed by freeze drying of the aqueous solution of the single strand oligonucleotide to produce a solid consisting essentially of the purified single strand oligonucleotide is a relatively expensive and time consuming process. Therefore, it would be an advance in the art if a process for producing nucleic acid duplexes were discovered that was less expensive and less time consuming.

### SUMMARY OF THE INVENTION

A primary benefit of the instant invention is a process for producing nucleic acid duplexes from purified single strand oligonucleotides, which process is less expensive and less time consuming than the prior art process. The central feature of the instant invention is the discovery that the prior art steps of dialysis and/or ultrafiltration of the solution of single strand oligonucleotide in the liquid chromatography eluant and the freeze drying to produce a solid consisting essentially of the purified single strand oligonucleotide can be eliminated without unduly increasing likelihood of microbial contamination during storage.

More specifically, the instant invention is a process for the preparation of at least one nucleic acid duplex, comprising the steps of: (a) providing a first single strand oligonucleotide in a crude solution, preferably by synthesizing a first single strand oligonucleotide to produce a crude solution of the first single strand oligonucleotide; (b) purifying the first single strand oligonucleotide from the crude solution by first liquid chromatography to produce a solution of purified first single strand oligonucleotide in a first liquid chromatography eluant; (c) providing a second single strand oligonucleotide in a crude solution, which second single strand has a complementary sequence or partial complementary sequence to the first single strand oligonucleotide, preferably by synthesizing a second single strand oligonucleotide to produce a crude solution of the second single strand oligonucleotide; (d) purifying the second single strand oligonucleotide from the crude solution by second liquid chromatography to produce a solution of purified second single strand oligonucleotide in a second liquid chromatography eluant; (e) mixing the solution of purified first single strand oligonucleotide in the first liquid chromatography eluant with the solution of purified second single strand oligonucleotide in the second liquid chromatography eluant so that the first and second single strand oligonucleotides can complex to form a nucleic acid duplex in the mixture of first and second liquid chromatography eluants.

Optionally, the instant invention may further comprise one or more of the following additional steps: 1) Storing at least one of the chromatography eluants, preferably for 24 hours or more, preferably less than 2 years, more preferably less than 1 year (provided for long storage times refrigeration is recommended) before mixing with the other single strand. Preferably, if the oligo strand is a DNA strand, the eluant has a pH greater than about 10 during the storage. Alternatively for DNA, or for an RNA strand, preferably the eluant contantains an antimicrobial agent (i.e. an inhibitor of microbial growth). The antimicrobial agent may be any known material which inhibits microbial growth in solutions of oligonucleotides. The antimicrobial agent may be an organic solvent such as acetonitrile or lower alcohols (e.g. methanol, ethanol, isopropanol). 2) Separating the nucleic acid duplex from the mixture of first and second liquid chromatography eluants by ultrafiltration or dialysis to produce a desalted aqueous solution of the nucleic acid duplex. 3) Freeze drying the desalted aqueous solution of the nucleic acid duplex to produce a solid comprising the nucleic acid duplex. 4) One or more additional single strand oligonucleotides may be provided and purified as was done for the first two strands. These additional single strand oligonucleotides are complementary to one of the first two oligonucleotide strands or to each other. These strands may be stored as noted above. In the mixing step, these additional single strand oligonucleotides are also added to provide a mixture of two or more different oligonucleotide duplexes.

In a related embodiment, the instant invention is a nucleic acid duplex made by such a process.

By complementary sequence as used herein is meant two strands are such that the bases are arranged in the two strands such that the strands can align and form a duplex by hydrogenation bonds of a base on one strand with a base on the other strand at multiple locations along the strands.

### DETAILED DESCRIPTION

The instant invention is a process for the preparation of a nucleic acid duplex comprising five actions or steps. The first step is to synthesize a first single strand oligonucleotide to produce a crude solution of the first single strand oligonucleotide. The second step is to purify the first single strand oligonucleotide from the crude solution of the first step by first liquid chromatography to produce a solution of purified first single strand oligonucleotide in a first liquid chromatography eluant. The third step is to synthesize a second single strand oligonucleotide to produce a crude solution of the second single strand oligonucleotide. The fourth step is to purify the second single strand oligonucleotide from the crude solution by second liquid chromatography to produce a solution of purified second single strand oligonucleotide in a second liquid chromatography eluant. The fifth step is to mix the solution of purified first single strand oligonucleotide in the first liquid chromatography eluant with the solution of purified second single strand oligonucleotide in the second liquid chromatography eluant so that the first and second single strand oligonucleotides can complex to form a nucleic acid duplex in the mixture of first and second liquid chromatography eluants. These steps may occur in any order provided a step that uses a result or outcome of another step must occur after that other step. For example, the fifth step must occur after the other four steps; the second step must follow the first step and the fourth step must follow the third step; however, steps three and four could precede, follow, or be performed concurrently with the first and/or second steps; steps one and three could precede steps two and four; etc.

The single strand oligonucleotides should be provided in a crude solution, preferably by synthesis by any known method. The crude oligonucleotide solution will contain the oligonucleotide having the desired length and targeted sequence and water. The solution is also likely to contain shorter and longer oligonucleotides than the desired target, potentially protecting groups and deprotection agents (e.g. ammonia) and other compounds that are residual from the synthesis operation. A preferred synthesis technique for the single strand oligonucleotides (the above-mentioned first and third steps) is the well known solid phase synthesis, as taught, for example, by Gait, OLIGONUCLEOTIDE SYNTHESIS, A PRACTICAL APPROACH, ISBN 0-904147-74-6. The single strand oligonucleotides can be of any type, for example and without limitation thereto, natural or synthetically modified, ribo or deoxyribonucleic acid structure wherein modifications may optionally be made to the bases by, for example and without limitation thereto, diaminopurine or 7-deaza-guanosine or the sugar moiety ex 2′-O-methyl, 2′-fluoro, or 2′-O-methoxyethyl and wherein the internucleotide linkages may be phosphodiester, phosphorothioate, phosphoramidate or phosphorothioamidate linkage or mixtures of such linkages. The first and/or second single strand oligonucleotides can optionally be capped or conjugated.

The crude solution of single strands of the target oligonucleotide are purified by liquid chromatography. The specific liquid chromatography system used in the instant invention (the above-mentioned second and fourth steps) is not critical and can be readily determined by a person skilled in the art. For example, when one performs the chromatography, the crude solution is placed on the column and then washed down with a buffered aqueous solution. Potentially one may add NaCl, 20 mmol caustic, or an organic solvent such as acetonitrile at this time. For DNA, one may use higher pH materials and have a resulting higher pH eluant, (e.g. greater than 10). For RNA, a pH closer to neutral is needed, hence the potential desire to use buffers such as sodium or potassium phosphate. In this regard, reference is made to, for example and without limitation thereto, “Therapeutic oligonucleotides: The state-of-the-art in purification techlonogies” by Sanghvi and Schulte (Current Opinion in Drug Discovery & Development 2004, Vol 7, No 6, p 765-776). As pointed out by Sanghvi and Schulte, simulated moving bed and membrane-based chromatographic or pseudo-chromatographic systems are new and emerging technologies for the purification of oligonucleotides and are encompassed herein under the term “liquid chromatography”. With regard to membrane chromatography purification of oligonucleotides, reference is also made to, for example and without limitation thereto, Lajmi et al., Organic Process Research & Development 2004, 8, 651-657. As noted above the conditions to inhibit microbial growth (high pH and/or antimicrobial agent) may be added in the course of chromatography or potentially could be present in the crude solution.

If not, the pH may be raised (for DNA) and/or an antimicrobial agent added to the eluant after chromatography.

The complexation of the first and second single strand oligonucleotides in the above detailed fifth step may require an adjustment of the pH of the solution of purified first single strand oligonucleotide in the first liquid chromatography eluant and/or the step of adjusting the pH of the solution of purified second single strand oligonucleotide in a second liquid chromatography eluant before the above-mentioned fifth step, to maximize the production of the nucleic acid duplex. And, it should be understood that the pH of the mixture of the above-mentioned fifth step can also be adjusted to maximize the production of the nucleic acid duplex. The specific pH employed will depend on the specific duplex being produced but usually the pH will be in the range of from about five to about ten. The first and/or second liquid chromatography eluants may contain water-miscible organic solvents such as acetonitrile and such eluants may be used directly in the above-mentioned fifth step of duplex formation. In addition, the presence of an organic solvent in either of the liquid chromatography eluants can serve to inhibit biological growth during storage and handling operations.

Preferably, the mixture of the above-mentioned fifth step is heated and then cooled (preferably to about forty to ninety degrees Celsius and then back to about twenty degrees Celsius) to anneal the nucleic acid duplex. The process of the instant invention can be used to produce a ribonucleic acid-ribonucleic acid duplex, a deoxyribonucleic acid-ribonucleic acid duplex or a deoxyribonucleic acid-deoxyribonucleic acid duplex. The concentration of each of the first and second single strand oligonucleotides in the above-mentioned fifth step is preferably in the range of from about 0.05 to about 5 wt % of the mixture.

The nucleic acid duplex can be purified from the mixture of first and second liquid chromatography eluants by ultrafiltration or dialysis to produce a desalted aqueous solution of the nucleic acid duplex. If the liquid chromatography eluant comprises an organic buffer such as triethylamine acetate or a different counterion than desired in the final duplex, then diafiltration can be employed to carry out the desired exchange of counterion. Diafiltration is also effective for removal of any water-miscible organic solvents present in the mixture of first and second liquid chromatography eluants. Freeze drying of the aqueous solution of the nucleic acid duplex can be employed to produce a solid comprising the nucleic acid duplex.

In a related embodiment, the instant invention is a nucleic acid duplex made according to the above-described process. Preferably, the nucleic acid duplex of the instant invention comprises more than ten but fewer than one hundred base pairs. More preferably, the nucleic acid duplex of the instant invention comprises more than ten but fewer than thirty base pairs.

### EXAMPLE 1

Sense and antisense single strand 20 base deoxy-oligonucleotides with phosphorothioate backbones were synthesized by solid phase synthesis using standard phosphoramidite chemistry to produce crude solutions of the first and second (or sense and antisense) single strand oligonucleotides which are then purified by preparative ion exchange chromatography. A high pH sodium chloride gradient eluant was used to elute each from the chromatography column packed with Source 30Q brand stationary phase (from Amershan Biosciences division of GE Healthcare, Waukesha Wis.). Fractions of the eluting first and second single strand oligonucleotides were collected and analyzed by ion pair-reverse phase chromatography. The fractions containing single strand oligonucleotide having a purity of greater than 90 area % were combined. The solution of purified first single strand oligonucleotide in the first liquid chromatography eluant weighs 2.2 killograms and contains 0.3 wt % of the first single strand oligonucleotide. The solution of purified second single strand oligonucleotide in the second liquid chromatography eluant weighed 2.9 killograms and contained 0.2 wt % of the second single strand oligonucleotide. The solutions were combined at room temperature in a stirred 5 liter reactor then heated to sixty degrees Celsius for 2 hours and then cooled back to room temperature to produce a nucleic acid duplex. Analysis by ion exchange chromatography showed that less than 4 area % of the chromatographic response is unreacted single stranded oligonucleotides and that 96 area % of the chromatographic response is the desired nucleic acid duplex. The duplex solution was then concentrated using a regenerated cellulose ultra-filtration cassette (0.1 m²) and diafiltered with USP water. The desalted solution of duplex was then freeze dried to yield 13 grams of dry product. Analysis of the dry product by ion exchange high performance liquid chromatography showed the product contains less than 1 area % of unreacted single strand oligonucleotides and more that 99 area % of the nucleic acid duplex.

### EXAMPLE 2

Crude solutions of the first and second (or sense and antisense) single strand oligonucleotides were synthesized by solid phase synthesis using standard phosphoramidite chemistry and then passed through liquid chromatography. The single strand solutions were retained for about 12 days under refrigeration. The solution (4 kg) of purified oligonucleotide of 21 bases in length and composed of ribose and 2′-O-methyl ribose units in phosphate buffered sodium chloride and approximately 5% acetonitrile was loaded to an ultra-filtration system with a 1K polyethersulfone membrane and concentrated to approximately 200 mL. A solution (1.7 kg) of a complementary strand of purified ribonucleotide was added to the retained solution in the ultrafiltration unit, mixed. A sample of the mixture was removed and analyzed by ion exchange chromatography to measure the relative amounts of each oligonucleotide. The analysis showed composition of the mixture to be 44.6 area % of the first oligonucleotide to 41.5 area % of the second. Additional solution of the second oligonucleotide was added, mixed and analyzed until the difference in area % for the two oligonucleotides was 1 area %. The ultra filtration was then continued to concentrate the solution to about 400 mL. To remove the acetonitrile, buffer and salt the mixture was diafiltered with deionized water. The retentate was recovered and filtered through a 0.2 um filter and freeze dried to yield 6.4 grams of duplexed oligonucleotide. Analysis by size exclusion chromatography showed the product to be 96 area % duplex. The thermal dissociation (Tm) of the duplex was measured at 69.8° C.

### EXAMPLE 3

Solutions of sense (62 OD/mL) and antisense (59 OD/mL) oligoribonucleotides (21 mers) recovered from preparative ion exchange chromatography of the individual oligos were stored for about 16 days and then combined in a ratio that would generate a one to one duplex. Two hundred twenty five grams of sense solution (phosphate buffered sodium chloride and 10% acetonitrile) was mixed with 266 grams of antisense solution. The mixture was analyzed by ion exchange HPLC to determine the ratio of the two single strands and then additional sense strand was added in aliquots and reanalyzed until the ratio of the single strands was essentially 1:1. After the addition of an additional 13.5 grams of sense solution, analysis showed the 16.3 area % sense and 16.6 area % antisense present under partial denaturing conditions. The mixture was then split in half. One half was concentrated using a 1K polyethersulfone membrane, diafiltered with deionized water and freeze dried. The other half of the duplex solution was heated to 85° C. and held there for 15 minutes then cooled to room temperature. This solution was then treated in the same manner as the first solution and concentrated using an ultra filtration membrane, diafiltered with water and freeze dried. Analysis of the isolated duplexes by ion exchange chromatography showed the purity of the duplexes to be 82 area % and 79 area % (with heating).

### CONCLUSION

While the instant invention has been described above according to its preferred embodiments, it can be modified within the spirit and scope of this disclosure. This application is therefore intended to cover any variations, uses, or adaptations of the instant invention using the general principles disclosed herein. Further, the instant application is intended to cover such departures from the present disclosure as come within the known or customary practice in the art to which this invention pertains and which fall within the limits of the following claims.

What is claimed is:

1. A process for the preparation of at least one nucleic acid duplex, comprising the steps of: (a) providing a first single strand oligonucleotide in a crude solution; (b) purifying the first single strand oligonucleotide from the crude solution by first liquid chromatography to produce a solution of purified first single strand oligonucleotide in a first liquid chromatography eluant; (c) providing a second single strand oligonucleotide which has a complementary or partially complementary sequence to the first single strand oligonucleotide in a crude solution; (d) purifying the second single strand oligonucleotide from the crude solution by second liquid chromatography to produce a solution of purified second single strand oligonucleotide in a second liquid chromatography eluant; (e) mixing the solution of purified first single strand oligonucleotide in the first liquid chromatography eluant with the solution of purified second single strand oligonucleotide in the second liquid chromatography eluant so that the first and second single strand oligonucleotides can complex to form a nucleic acid duplex in the mixture of first and second liquid chromatography eluants.

2. The method of claim 1 wherein the step of providing a single strand oligonucleotide comprises synthesizing the single strand oligonucleotide.

3. The method of claim 1 wherein at least one of the chromatography eluants is stored for more than 24 hours.

4. The method of claims 1 or 3 wherein the eluant has a pH of greater than and the single strand is DNA.

5. The method of claim 1 or 3 wherein the eluant comprises an antimicrobial inhibitor.

6. The method of claim 5 wherein the antimicrobial inhibitor is an organic solvent.

7. The method of claim 6 wherein the organic solvent is acetonitrile or a lower alcohol.

8. The process of claim 1, further comprising the step of separating the nucleic acid duplex from the mixture of first and second liquid chromatography eluants by ultrafiltration to produce an aqueous solution of the nucleic acid duplex.

9. The process of claim 8, further comprising the step of freeze drying the aqueous solution of the nucleic acid duplex to produce a solid comprising the nucleic acid duplex.

10. The process of claim 1 or 4, further comprising the step of adjusting the pH of the solution of purified first single strand oligonucleotide in a first liquid chromatography eluant before step (e).

11. The process of claim 1 or 4, further comprising the step of adjusting the pH of the solution of purified second single strand oligonucleotide in a second liquid chromatography eluant before step (e).

12. The process of claim 1 or 4, further comprising the steps of adjusting the pH of the solution of purified first single strand oligonucleotide in a first liquid chromatography eluant and the step of adjusting the pH of the solution of purified second single strand oligonucleotide in a second liquid chromatography eluant before step (e).

13. The process of claim 1, farther comprising adjusting the pH of the mixture in step (e).

14. The process of claim 1, further comprising in step (e) heating and then cooling the mixture of first and second liquid chromatography eluants to anneal the nucleic acid duplex.

15. The process of claim 1, wherein the nucleic acid duplex is an RNA-RNA duplex.

16. The process of claim 1, wherein the concentration of the first and second single strand oligonucleotides in step (e) is in the range of from about 0.05 to about 5 wt % of the mixture.

17. The process of claim 14, wherein the heating and cooling temperature range is from about ninety to about twenty degrees Celsius.

18. The process of any of claims 10-12, wherein the adjusted pH is in the range of from about five to about ten.

19. The process of any of the preceding claims further comprising purifying a third single strand oligonucleotide in crude solution by liquid chromatography to form a third eluant said third single strand has complementary sequence or partially complementary sequence to one of the first two single strands is purified and mixing the third eluant with the first two eluants in the mixing step.

20. The process of any of the preceding claims further comprising purifying a third single strand oligonucleotide in crude solution by liquid chromatography to form a third eluant and purifying a fourth single strand oligonucleotide in crude solution by liquid chromatography to form a fourth eluant which fourth single strand oligonucleotide has a complementary sequence or partially complementary sequence to the third single strand oligonucleotide, and in the mixing step mixing the first, second, third and fourth liquid chromatography eluants.

21. A nucleic acid duplex made according to the process of any of claims 1-20.
